(19) United States
(12) Patent Application Publication
(10) Pub. No.: US 2016/0000415 A1
(43) Pub. Date: Jan. 7, 2016

(54) MULTIPLE-TISSUE FNA SAMPLING

(71) Applicant: RAMBAM HEALTH CORPORATION, Haifa (IL)

(72) Inventors: Ziv BELSKY, Haifa (IL); Gilad HIZKIYAHU, Givat Ela (IL); Jesse LACHTER, Haifa (IL)

(73) Assignee: Rambam Health Corporation

(21) Appl. No.: 14/772,364

(22) PCT Filed: Mar. 4, 2014
(86) PCT No.: PCT/IB2014/059420

§ 371 Date: Sep. 2, 2015

Related U.S. Application Data
(60) Provisional application No. 61/772,059, filed on Mar. 4, 2013.

(51) Int. Cl. A61B 10/04; A61B 10/02

(52) U.S. Cl. CPC A61B 10/04; A61B 10/0233; A61B 2010/0225; A61B 2010/045

(57) ABSTRACT

An apparatus, device and method are provided for endoscopically extracting multiple tissue samples from multiple locations without needing to completely retract the apparatus or device from the target tissue in the process. In some embodiments the device includes a sheath for delivering the device through a body lumen wall, an outer needle, an inner needle extendable from and retractable into the outer needle, the inner needle having a rotational feature to enable sequential entry of the inner needle at multiple angles relative to the outer needle; and a rotational control mechanism for controlling rotation of the inner needle thereby enabling tissue extraction from multiple locations in a target tissue, without sequentially exiting the body lumen wall.

### FIELD OF THE INVENTION

The present invention is related to medical instrumentation, and more specifically to endoscopic tissue collection.

### BACKGROUND

Tissue/Fluid sampling is used in a number of fields, including gastroenterology, cardiology, oncology, radiology, ophthalmology, histology, neurology and neurosurgery, internal medicine, and renal specialties. Such samples are commonly used in the performance of biopsies, or the removal of tissue samples. Biopsies are typically used to help in diagnosing of, for example, a variety of diseases. However it is often necessary to take multiple samples in order to increase the accuracy of the diagnosis, since a single tissue sample my not represent the entirety of the area, organ, or lesion from which the sample is extracted. Unfortunately the taking of multiple samples can be time consuming, uncomfortable or painful, and may also increase risks of infection.

Gastroenterologists, surgeons, and other physicians commonly obtain tissue samples for biopsy when examining interior parts of the body using an endoscope. Modern endoscopes are usually flexible instruments comprising a fiber optic viewing system and a tubular channel through which biopsy forceps can be passed to obtain the samples. Some prior art biopsy forceps are designed to obtain a single small piece of tissue on each passage through the endoscope. Such single pass forceps, however, are time consuming to use since clinicians frequently require multiple biopsies of a diseased area in order to gather adequate pathological or other scientific information. The instrument must be passed in and out of the endoscope for each biopsy specimen, and the findings through such procedures are generally limited to visual inspection inside the GI tract.

Fine-needle aspiration biopsy (FNAB, FNA or NAB), or fine-needle aspiration cytology (FNAC), is a common diagnostic procedure used to investigate superficial lumps or masses. In this technique, a thin, hollow needle is typically inserted into the tissue for sampling of cells that, after being stained, may be examined under a microscope. There may also be a cytology exam of aspirate or histological tissue. Although needle aspiration biopsy is typically safer and less traumatic than an open surgical biopsy, common complications include bruising and soreness. Further, there is a risk, because the biopsy is very small, that the problematic cells will be missed, resulting in a false negative result. However since FNA is generally implemented through the skin to tissue or organs below the skin, it cannot adequately access tissue or organs covered by bones or under other organs etc.

In addition, the disadvantage with many currently used devices is that the device must be withdrawn from patient after a single biopsy sample is obtained. Accordingly, it is desirable to extract a number of tissue samples in a minimally invasive manner.

EUS-FNA is fine needle aspiration during Endoscopic UltraSound, using an ultrasound equipped endoscope. The endoscope is inserted into the GI tract and its distal end is placed near the desired target organ. The ultrasound detector, integral to the device, is activated, and is used to scan organs and tissue adjacent to the GI tract but external to it. When a biopsy is deemed necessary by the physician, a special EUS-FNA needle is passed through the endoscope working channel, punches through the GI tract wall and is guided, under ultrasound, to reach the desired area in the body and obtain a tissue sample for cytological or histological evaluation. EUS-FNA thereby enables inspection of tissues or organs outside the GI tract. However, making a hole in the GI tract wall carries several risks, including the risk of excessive bleeding, and having material from the GI tract leak through such a hole, creating irritation and probably inflammation in the body.

It is therefore of much value to use the smallest needle possible, and to create as few holes as possible in the GI tract wall. Accordingly, a physician generally tries to aim the needle at the target area for sampling when executing EUS-FNA procedures. Current EUS FNA needles generally have linear control only, meaning they can only be inserted or withdrawn along their main axis and have no other intrinsic means of control over the area reached. Only the endoscope elevator can affect the angle that the needle assumes entering the tissue relative to the endoscope itself. However even this elevator generally fails to give the physician enough control over the needle tip position to allow them to reach the location they think is best to draw a sample from. Furthermore, using different elevator angles requires multiple needle insertions (at different elevator angles) and consequently several holes in the GI tract, with the associated risks mentioned above. Moreover, repeated needle insertion and biopsy attempts cause more damage to the tissue in the area that the physician is trying to reach, and in many cases requires more expensive needles to be used, due to damage to the needle itself when it is used several times.

### SUMMARY

There is provided, in accordance with an embodiment of the present invention, an apparatus, system, and method for tissue extraction from multiple locations without needing to extract the needle back through a body lumen wall hole (such as a GI tract wall hole) initially created.

According to some embodiments, a device is provided for endoscopically extracting multiple tissue samples sequentially from a subject, comprising: a sheath; an optionally flexible outer needle; and an inner needle extendable from and retractable into the outer needle having a rotational feature to enable sequential entry of the inner needle at multiple angles relative to the outer needle; and a rotational control mechanism for controlling rotation of the inner needle thereby enabling tissue extraction from multiple locations in a target tissue without sequentially exiting through the lumen wall.

In some embodiments, the rotational control mechanism is located on a base associated with the outer needle.

In some embodiments, the inner needle is comprised of a shape memory material such that it forms a pre-configured line of curvature when extended from the outer needle.

In some embodiments, the shape memory material is a biocompatible nickel titanium alloy (nitinol).

In some embodiments, the outer needle is adapted to maintain the inner needle in a straight configuration when retracted within the outer needle.

In some embodiments, a multiple control mechanism is provided to independently maneuver inner and outer needles.

In some embodiments, a retractable stylet is provided within the inner needle.

According to some embodiments, a device is provided for obtaining multiple biopsy samples, optionally substantially simultaneously, from a body cavity, comprising:

an outer sheath; multiple inner needles extendable from and retractable into the sheath and being adapted to penetrate a target tissue and to extract samples of the tissue, optionally substantially simultaneously, without sequentially exiting the target tissue, and a control mechanism for controlling the extension and retraction of the inner needles from the sheath.
In some embodiments, the control mechanism is located on a base associated with the needles.
In some embodiments, each needle has a pre-configured shape.
In some embodiments, the multiple needles having different pre-configured lines of curvature.
In some embodiments, at least one needle is straight and at least one needle has a pre-configured line of curvature.
In some embodiments, the control mechanism is adapted to extend the multiple needles simultaneously or individually from the external sheath.
In some embodiments, each needle is in communication with a distinct chamber for collection of a sample from that needle.
In some embodiments, each inner needle is substantially straight and a deflecting cap is provided on the outer sheath and/or each needle to deflect the needle at an angle upon exiting the sheath.
In some embodiments, multiple retractable stylets are provided within the inner needles.

According to some embodiments, a method is provided for obtaining multiple tissue samples through an endoscope, the method comprising (i) advancing an endoscopic device having an outer needle and a remotely rotatable inner needle through a body lumen wall, such as a GI tract, the inner needle having a pre-configured curvature and being retracted within the outer needle; (ii) upon reaching a first target tissue site, extending said inner needle from said outer needle to capture a tissue sample, said inner needle adopting its pre-configured curvature; (iii) at least partially retracting the inner needle within said outer needle; (iv) rotating angle of said inner needle; and (v) advancing the rotatable inner needle with a pre-configured curvature to a second or additional location in target tissue to capture a second or additional tissue sample.

According to some embodiments, a method is provided for obtaining multiple tissue samples, optionally substantially simultaneously, through an endoscope, the method comprising (i) advancing an endoscopic device comprising an outer sheath with multiple inner needles through a body lumen wall, such as a GI tract, each of the internal needles optionally having a distinct pre-configured curvature and being retracted within the external sheath; (ii) upon reaching a target tissue site, extending one or more of the inner needles through the body lumen wall to multiple locations within a target tissue, to capture multiple tissue samples; and (iii) at least partially retracting one or more of the inner needles into said outer sheath.

The devices and methods of the present invention are particularly suitable for fine needle aspiration biopsies (FNA), especially fine needle aspiration during endoscopic ultrasound (EUS-FNA).

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIGS. 1A-1D are graphical illustrations of a rotatable, pre-curved coaxial EUS-FNA device, enabled to reach tissue at multiple targets substantially through a single entry hole in the target location/organ, according to some embodiments;

FIGS. 2A-2G are graphic illustrations of a multiple needle EUS-FNA device enabled to reach tissue at multiple locations substantially simultaneously, according to some embodiments;

FIG. 3 is a is a flowchart illustrating a series of operations or processes that may be implemented to enable tissue extraction from multiple locations, using a EUS-FNA device with a rotatable tissue capture needle, as illustrated in FIGS. 1A-1D; and

FIG. 4 is a flowchart illustration showing a series of operations or processes that may be implemented to enable substantially simultaneous tissue extraction from multiple locations, using a EUS-NFA device with multiple tissue capture needles, as illustrated in FIGS. 2A-2G.

It will be appreciated that for simplicity and clarity of illustration, elements shown in the drawings have not necessarily been drawn to scale. For example, the dimensions of some of the elements may be exaggerated relative to other elements for clarity. Further, where considered appropriate, reference numerals may be repeated among the drawings to indicate corresponding or analogous elements throughout the serial views.

### DETAILED DESCRIPTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

In accordance with some embodiments of the present invention, there is provided an FNA needle able to reach multiple points in a target tissue when it is inserted once through a hole made in the GI tract wall, obviating the need to extract the needle back through the GI tract wall during a procedure. In a first embodiment, different locations for tissue sampling may be reached sequentially in a single entry into a target organ. In a second embodiment different locations for tissue sampling may be reached substantially simultaneously.

According to some embodiments of the present invention, tissue removal from multiple locations may be enabled within a substantially single EUS-FNA procedure, by using a rotatable bending shaped needle adapted to extract tissue in multiple locations.

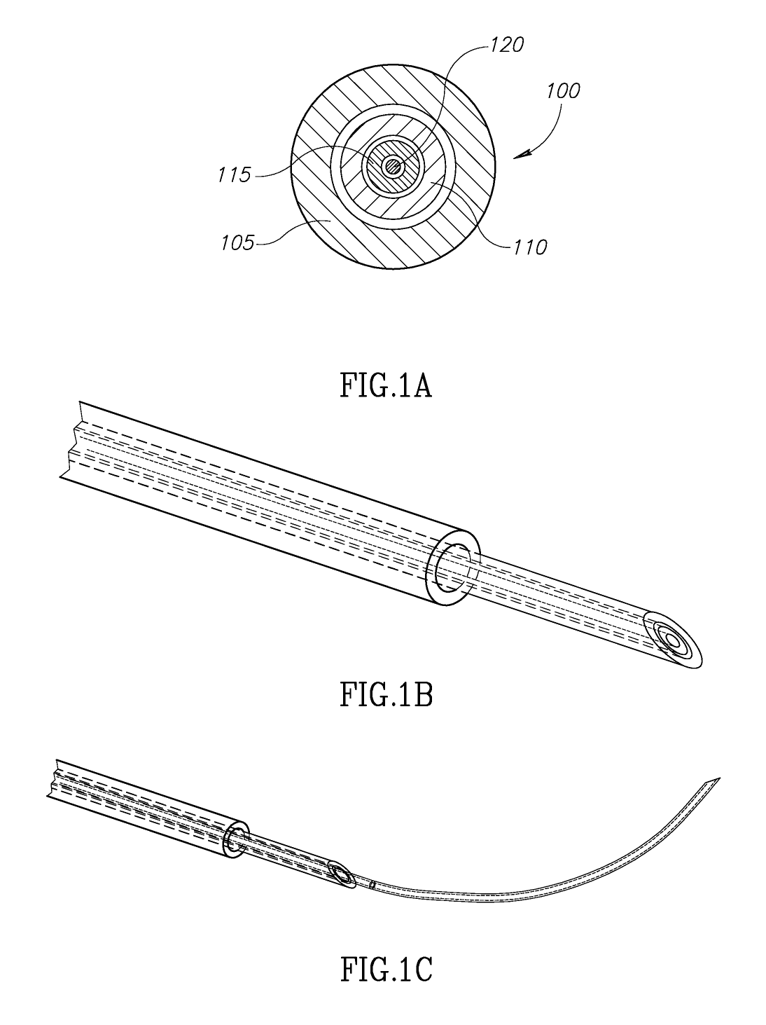

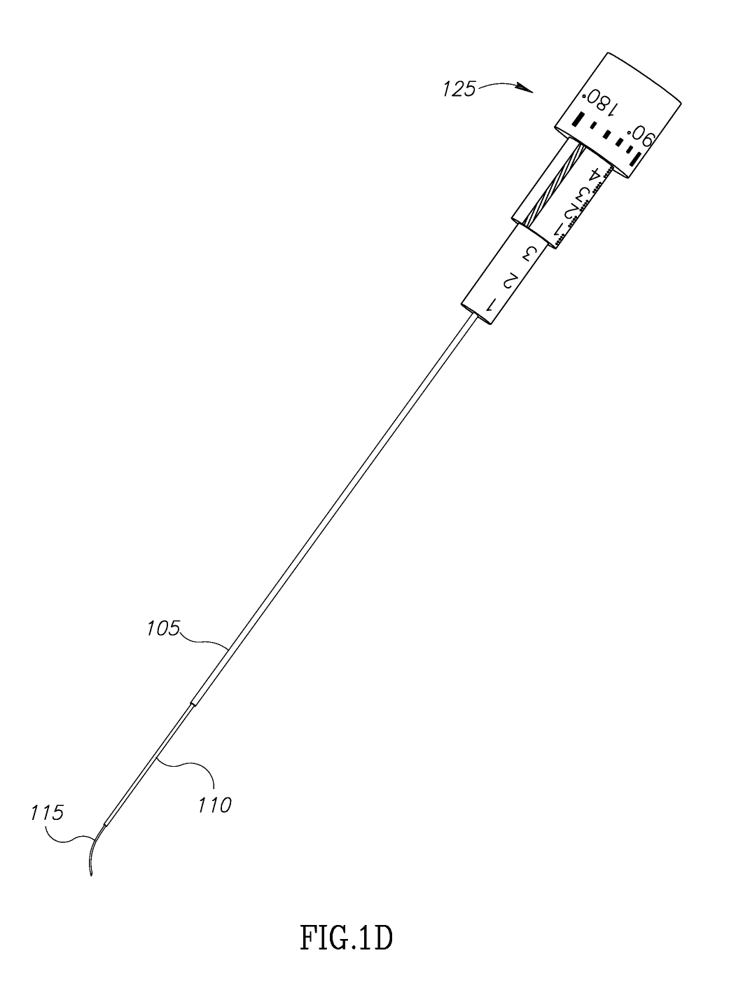

Reference is now made to FIGS. 1A-1C which are graphical illustrations of a rotatable, pre-curved coaxial needle device 100, for endoscopically extracting multiple tissue samples sequentially from a subject, according to some embodiments. In some embodiments the needle may be made from a flexible metal or constructed at least partially from Nitinol or another shape based material. Of course, other suitable materials may be used.

As can be seen, the device components may include an external sheath 105, optionally flexible, for delivering the device through a body lumen wall, housing an optionally flexible outer needle 110, suitable to be delivered endoscopically. In one example, sheath 105 has an outer diameter of ˜2.1 mm and an inner diameter of ˜1.3 mm. Of course other dimensions may be used. In one example outer needle 110 may have a size of ˜19 ga, and have an OD of ˜1.076 mm and IN of ˜0.68 mm. Of course other dimensions may be used. In addition, there is an inner needle 115, extendable from and retractable into the outer needle, wherein the inner needle has a rotational feature to enable sequential entry of the inner needle at multiple angles relative to the outer needle. In one example, the inner needle may be constructed from a flexible material, and in some cases, at least partially from a shape memory material such that it forms a pre-configured line of curvature when extended from the outer needle. In some embodiments the shape memory material is a biocompatible nickel titanium alloy (nitinol). In some examples, the inner needle may have a size of ˜25 ga, and have an OD of ˜0.52 mm and an IN of ˜0.26 mm. Of course other dimensions may be used. In addition, there may be a stylet 120. In one example, stylet 120 may have a diameter of ˜0.16 mm. Of course other dimensions may be used. In addition, the device includes a rotational control mechanism for controlling rotation of the inner needle thereby enabling tissue extraction from multiple locations in a target tissue, without exiting a target organ. In some examples, the rotational control mechanism is located on a base associated with the outer needle. In some embodiments, needle device 100 may be at least 100 cm long (i.e., to be suitable to be used with an endoscope, whether less or more than 100 cm) and may in its entirely to be able to substantially bend, for example, to enable forming a circle with a diameter of 30 cm (or more or less).

In further embodiments, the outer needle is adapted to maintain the inner needle in a straight configuration when retracted within the outer needle.

In additional embodiments, a multiple control mechanism is provided to independently maneuver both the inner and outer needles.

In still further embodiments, a retractable stylet is provided within the inner needle.

In one or more of the above embodiments, the inner needle is adapted for therapy delivery.

As can be seen in FIG. 1D, the device may also have an FNA system handle 125 with added knob(s) or control dial(s) for internal needle insertion and rotation control. In the current example, as can be seen in FIG. 1D, a Nitinol needle may have a pre-configured curvature, for example, like the curvature of the circumference of a 3 cm diameter circle, however, other suitable shapes or forms may also be used. In some embodiments the pre-shaped inner needle 115 is adapted to be rotatable while inside an outer needle 110, so that when pushed out of a sheath 105, inner needle 115 will curve in a different direction toward the target tissue. Outer needle 110 is adapted to be sufficiently hardened to enable the curved inner needle to be maintained in a substantially straight position when inside the outer needle. Further, the relative angle of the inner needle 115 and outer needle 110 should be indicated on the handle 125, to help the practitioner maneuver the needle to different targets. Further, signing or angle measurement indications may be provided on handle 125 to indicate the relative angle between inner needle 115 and outer needle 110, relative to a pre-set reference angle. In some embodiments, inner needle 115 may be filled by an extractable stylet 120 that, when fully inserted within inner needle 115 reaches its tip, to protect the inner needle entry point so it only grabs target tissue when stylet 120 is retracted.

In some embodiments, the end of the hollow inner needle may be attached to a connector on handle 125 (e.g., on the middle of back part) that allows connection of the inner needle to a suction source, to enable suction to be implemented from the tip of the needle.

In some embodiments, a multiple-level control mechanism may be used to maneuver outer and inner needles in the EUS-FNA procedure, to enable rotation of the inner needle while inside the outer needle. For example, two or more handles or guide elements may be used to control both the inner needle and outer needle linear position relative to the external sheath and also the inner needle angle relative to the outer needle during a procedure. As can be seen in FIG. 1B, the outer needle may be extended to penetrate the tissue, thereby reaching a desired location in the sample area to be extracted. Further, as can be seen in FIG. 1C, the inner needle may be extended as deeply as needed into the target tissue (e.g., the pancreas). As can be seen, the natural bending of the needle can occur during tissue penetration. Further, the inner needle may be retracted, optionally all the way back into the outer needle, following a tissue sampling, which appears similar to the device shown in FIG. 1B. Further, one or more additional samplings may be conducted without extracting the EUS-FNA device, by rotating the inner needle (e.g., 45°/90°) using the base knob and then extending the inner needle again, as may be necessary. The natural bending may herein occur during tissue penetration on a different plane, as can be seen. The rotation and re-execution may be conducted multiple times. Following completion of the one or more tissue extraction procedures, the device may be retrieved or extracted, and flushed or cleaned using, for example, air or fluid, to extract the tissue samples for testing. Needles may be discarded or sterilized for further use. Optionally, after stylet removal and prior to sampling, a suction source may be applied to the base of inner needle 115 through a standard connector in handle 125 (not shown), to help harvest the tissue sample and store it within the needle until flushing, as is known in the art and commonly practiced with existing EUS-FNA needles.

In other embodiments of the present invention, multiple location tissue removal is enabled using a tissue sample removal device with multiple needles adapted to acquire tissue from multiple locations simultaneously.

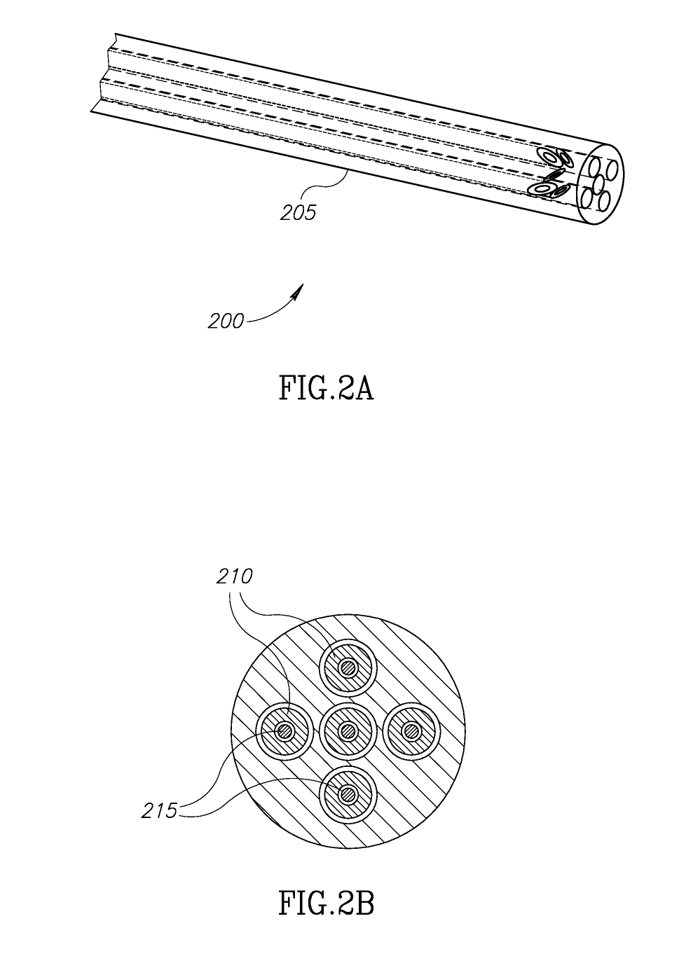

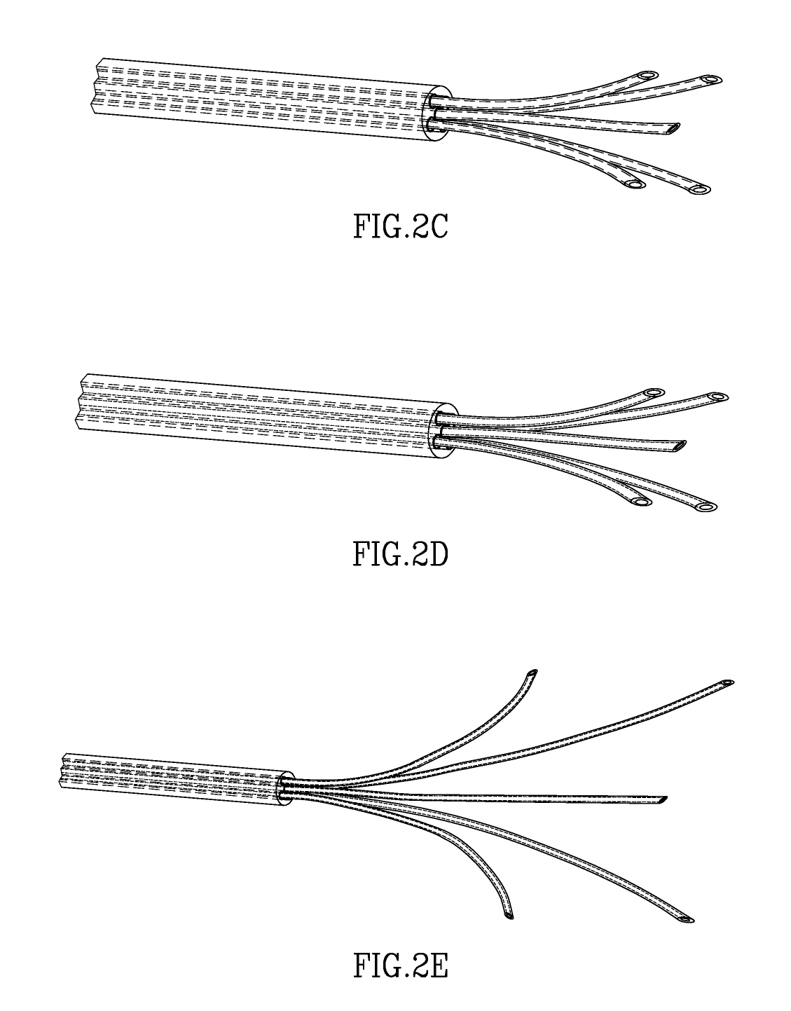

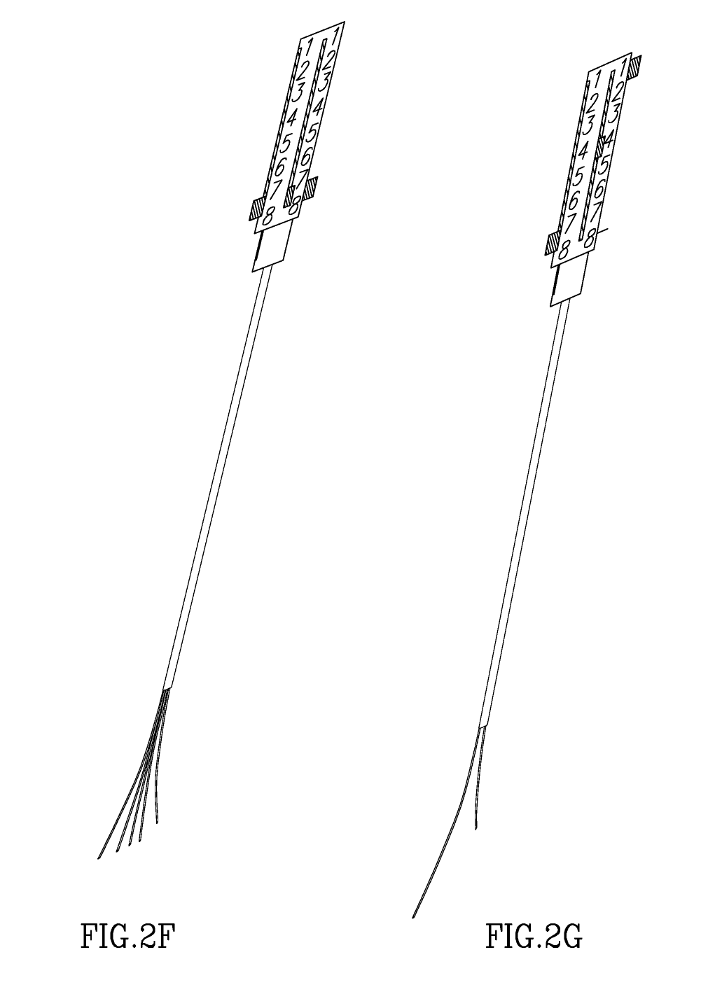

Reference is now made to FIGS. 2A-2B which are graphic illustrations of a multiple needle/capture EUS-FNA device 200 enabled to remove tissue from multiple locations simultaneously, according to some embodiments. As can be seen in FIGS. 2A-2B, device 200 components include an external sheath 205 that holds multiple inner needles 210, optionally with inner Stylets 215, to prevent entry of tissue materials into needles until after the stylets are removed. In some embodiments the respective inner needles may be contained within distinct lumen (i.e. multiple inner lumen) to prevent inner needle buckling. In some embodiments, each inner needle has a pre-configured shape, and/or may have different pre-configured lines of curvature. In some embodiments, at least one needle is straight and at least one needle has a pre-configured line of curvature. In some embodiments, each needle is in communication with a distinct chamber for collection of a sample from that needle. In some embodiments, each inner needle is substantially straight and a deflecting cap is provided on the outer sheath and/or each needle to deflect the needle at an angle upon exiting the sheath. In some embodiments, multiple retractable stylets are provided within the inner needles.

In some embodiments, the control mechanism is located on a base associated with the needles. Further, the control mechanism may be adapted to extend the multiple needles simultaneously or individually from the external sheath.

FIGS. 2A-2B show one example of the inner design of such a FNA device, however other configurations may be used, with different scales, numbers of lumen etc. FIGS. 2C-E are graphical illustrations of an example of device 200 being deployed, wherein the different pre-configured curvatures of the respective inner needles can be seen, as they are advanced. In some embodiments device 200 may include multiple Inner needles 210, for example ±25 ga, which may be pre-curved or not. In one example, a 22 GA needle may be used, 3 needles around it of 25 GA size. In a further example a sheath 205 may have an OD of ±2.7 mm, and it may contain multiple lumen, for example 5 lumen of ±0.62 mm diameter each, or one central lumen of ±0.8 mm and 3 or 4 surrounding lumen of ±0.62 mm each. In some examples, inner needles may have an OD of ±0.52 mm and an IN of ±0.26 mm. Of course, other dimensions may be used. Device 200 may also include multiple stylets 215, within inner needles 210, one stylet within each needle. In some examples, stylet 215 may have a diameter of ±0.16 mm. In some embodiments, for example where pre-curving needles may not be effective, a deflecting cap that connects to the sheath may be provided, to enable protection of the entrance of the inner needle so as to minimize the entry of unwanted materials during a procedure. Of course, other dimensions may be used, and other numbers, sizes, types etc of needles may be used.

In some embodiments the device 200 may include multiple chambers, one for each inner capture needle, for simultaneous collection of samples. For example, the device may maintain separate containment and extraction areas and ports, to help a practitioner or tester know which location each sample was taken from.

Reference is now made to FIGS. 2F-2G, which are graphical illustrations of examples of further embodiments of device 200, which may include multiple handles or other controls for maneuvering the respective inner needles. As can be seen in the figures, the respective inner needles may be advanced simultaneously to the target tissue to acquire tissue samples. In other embodiments the respective inner needles may be individually advanced via the respective control dials or mechanisms, optionally with individual measurement panels to help the practitioner advance each inner mechanism to a preferred distance. FIG. 2G illustrates the scenario where only two inner details were advanced, and these were advanced to different distances, in accordance with the practitioners determination.

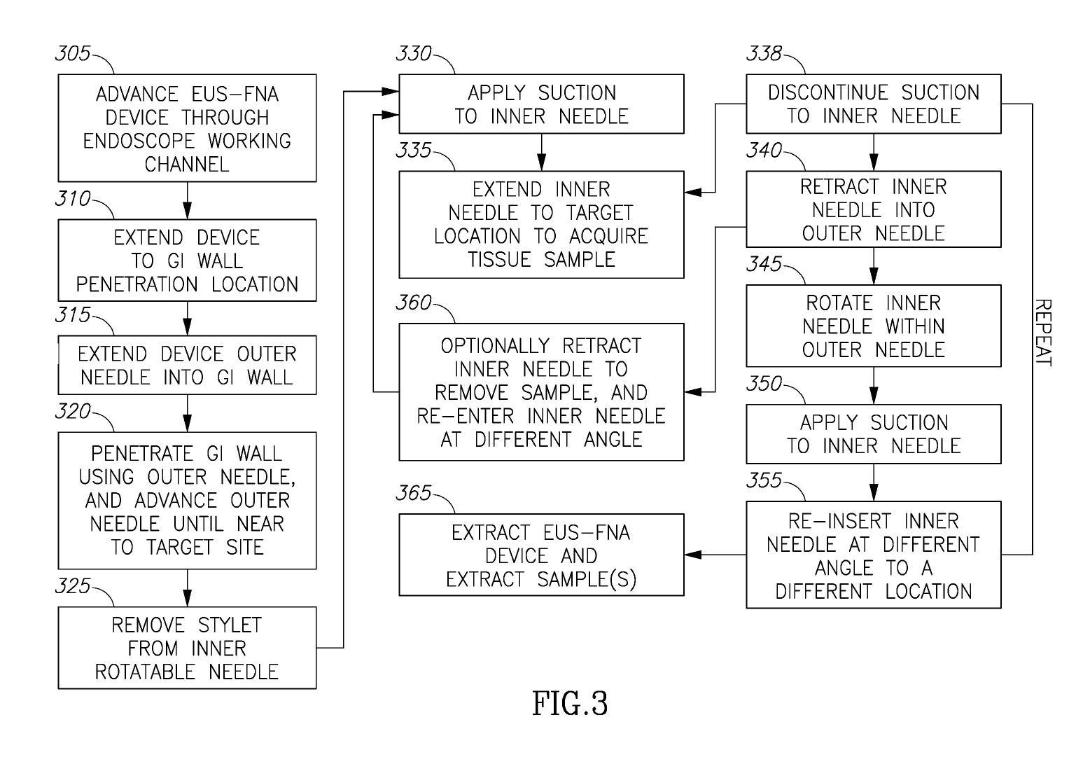

FIG. 3 schematically illustrates a series of operations or processes that may be implemented to enable tissue extraction from multiple locations, using an EUS-FNA device as illustrated in FIGS. 1A-1D. As can be seen in FIG. 3, at block 305 an EUS FNA device may be inserted into the working channel of an already positioned ultrasound endoscope (EUS). It is important to note that prior to use, EUS-FNA device is typically set so that both the outer and inner needles are fully retracted into the sheath, and a stylet is typically fully inserted into the inner needle. At block 310 the EUS-FNA device may be extended to a position where a practitioner wishes to penetrate the GI tract to access a target tissue or organ. At block 315 an endoscope having an outer needle and a rotatable inner needle is rotated through a wall of a body lumen, such as the GI wall. In some cases the inner needle has a pre-configured curvature, and is retracted within the outer needle. At block 320 the outer needle may penetrate the GI wall and continue to be advanced until near the target sampling site. At block 325 the stylet may be removed to enable the inner needle to be readied for tissue capture. At block 330 suction may be applied to the inner needle, to aid tissue capture. At block 335 the inner needle may be extended from the outer needle, to capture a tissue sample from a target location, wherein the inner needle adopts its pre-configured curvature, to enable tissue sample capture. While the needle is extended it may bend or curve in accordance with its natural or pre-configured properties, for example, along a pre-determined plane, thereby reaching the desired site for tissue sampling. At block 338 the suction to the inner needle may be discontinued. At block 340 the inner needle may be retracted at least partially back into the outer needle. At block 345 the inner needle may be rotated, for example between 10°-170° or more specifically between 45°-90°, using the base knob or other rotating device, within the outer needle. At block 350 suction may be re-applied to the inner needle. At block 355 the inner needle with a pre-configured curvature may be extended again to enable tissue penetration on a different plane or at a different angle, to enable tissue capture at a second (or additional) location. Steps 338-355 may be repeated numerous times to enable tissue capturing in the inner needle from multiple locations.

In some embodiments, at block 360, the inner needle may be retracted out of the patient, and the tissue samples may be flushed or otherwise removed from the needle for storage or analysis. Suction may then be applied at block 330, and the inner needle may be re-entered all the way until it extends from the outer needle, optionally at a second or alternative angle or position, at block 335 to continue with the work flow as before. At block 365 the EUS-FNA device may be extracted entirely and the sample(s) extracted typically by flushing the needle. The EUS-FNA device may optionally be cleaned/prepared for re-use. Any combination of the above steps may be implemented. Further, other steps or series of steps may be used.

In some embodiments, a variety of tissue grabbing, cutting or extraction elements may be used. Further, a variety of cleaning, flushing or extracting mechanisms may be used to release the tissue samples from the FNA device.

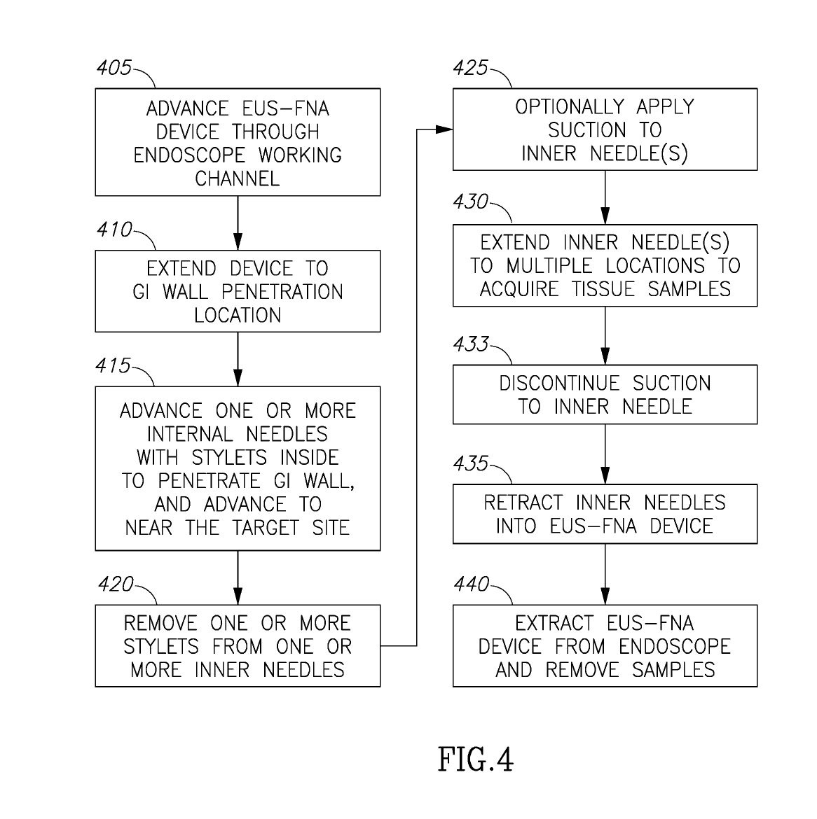

FIG. 4 schematically illustrates a series of operations or processes that may be implemented to enable tissue extraction from multiple locations substantially simultaneously, using an EUS-FNA device as illustrated in FIGS. 2A-2G. As can be seen in FIG. 4, at block 405 an EUS-FNA device may be inserted into an already positioned EUS endoscope and advanced through the endoscope working channel all the way towards the tip. At block 410 the needles may be advanced to the GI wall penetration location. At block 415 one or more needles, optionally having one or more pre-configured curvatures, may be further advanced to penetrate the GI wall and further advanced towards the target site. In the case where pre-curved needles are used, the pre-curving of the needles will cause them to spread out in the tissue and reach different locations. At block 420 one or more stylets may be removed from the inner needles. At block 425 suction may optionally be applied to one or more inner needles to help tissue capture. At block 430 one or more inner needles are extended as deeply as needed, to penetrate and acquire tissue samples. At block 433 suction to the inner needle may be discontinued. At block 435 the inner needles may be retracted into the device sheath. At block 440 the EUS-FNA device may be extracted from the endoscope, and the samples removed for further storage or analysis from the needles by way of flushing or any other method known in the art. In some cases sample extraction may require rinsing/dispensing the device. In some cases the EUS-FNA device may cleaned/prepared for re-use. Any combination of the above steps may be implemented. Further, other steps or series of steps may be used.

According to some embodiments, substantially straight inner needles may be used, optionally with needle caps (on the sheath) that deflects the needles at respective angles when they exit the sheath. In other embodiments alternative mechanical elements may be used to maneuver the inner needles at different angles.

According to some embodiments, one or more of the multiple inner needles may be individually maneuvered.

According to some embodiments of the present invention, multiple tissue collection methods may be used in the above described systems and methods.

In some embodiments brushing or drilling may be implemented, for example using a rough drilling stylet.

In still further embodiments a moving elevator mechanism may be used in conjunction with the described EUS-FNA device(s).

In other embodiments, the device may include multiple chambers for storage and optionally for simultaneous flushing/collection of samples. In some implementations the separate ports may be used to know where each sample has been taken from.

In some embodiments, different handles or control may be used to maneuver or control the different inner needles.

In further embodiments, therapeutic treatments or drug delivery may be implemented using the devices described above.

In further embodiments, elastography may be integrated during the tissue sampling procedure.

In further embodiments, needles of different length, strength, materials etc. may be used.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

What is claimed is:

1. A device for endoscopically extracting multiple tissue samples sequentially from
a subject, comprising:
a sheath for delivering the device through a body lumen wall;
an outer needle;
an inner needle extendable from and retractable into said outer needle, said inner needle having a rotational feature to enable sequential entry of said inner needle at multiple angles relative to said outer needle; and
a rotational control mechanism for controlling rotation of said inner needle thereby enabling tissue extraction from multiple locations in a target tissue, without sequentially exiting said body lumen wall.

2. The device as claimed in claim 1, wherein the rotational control mechanism is located on a base associated with the outer needle.

3. The device as claimed in claim 1, wherein the inner needle is comprised of a shape memory material such that it forms a pre-configured line of curvature when extended from the outer needle.

4. A device as claimed in claim 3, wherein the sheath, outer needle and/or inner needle are flexible enough to be suitable to be delivered endoscopically.

5. A device as claimed in claim 3, wherein the outer needle is adapted to maintain the inner needle in a straight configuration when retracted within the outer needle.

6. A device as claimed in claim 1, wherein a multiple control mechanism is provided to independently maneuver inner and outer needles.

7. A device as claimed in claim 1 further comprising a retractable stylet provided within the inner needle.

8. The device of claim 1, wherein said inner needle is adapted for therapy delivery.

9. A device for endoscopically extracting multiple tissue samples from a body cavity, comprising:
an outer sheath;
multiple inner needles extendable from and retractable into said outer sheath and being adapted to penetrate a target tissue and to extract samples of said tissue, optionally substantially simultaneously, without sequentially exiting a body lumen wall; and
a control mechanism for controlling the extension and retraction of said inner needles,
wherein said outer sheath and said inner needles are flexible enough to be suitable to be delivered endoscopically.

10. A device as claimed in claim 9, wherein the control mechanism is located on a base associated with said needles.

11. A device as claimed in claim 9 wherein each needle has a pre-configured shape.

12. A device as claimed in claim 11 wherein the multiple needles having different pre-configured lines of curvature.

13. A device as claimed in claim 11 wherein at least one needle is straight and at least one needle has a pre-configured line of curvature.

14. A device as claimed in claim 9, wherein the control mechanism is adapted to extend the multiple needles simultaneously or individually from the external sheath.

15. A device as claimed in claim 9, wherein each needle is in communication with a distinct chamber for collection of a sample from that needle.

16. A device as claimed in claim 9 wherein each inner needle is substantially straight and a deflecting cap is provided on the outer sheath and/or each needle to deflect the needle at an angle upon exiting the sheath.

17. A device as claimed in claim 9 further comprising multiple retractable stylets provided within the inner needles.

18. The device of claim 9, wherein said inner needle is adapted for therapy delivery.

19. A method for obtaining multiple tissue samples through an endoscope, the method comprising:
(i) advancing an endoscope having an outer needle and a rotatable inner needle through a wall of a body lumen, said inner needle having a pre-configured curvature and being retracted within the outer needle;
(ii) upon reaching a target tissue site, extending said inner needle from said outer needle to capture a tissue sample, said needle adopting its pre-configured curvature;
(iii) at least partially retracting said inner needle within said outer needle;
(iv) rotating angle of inner needle; and
(v) advancing said rotatable inner needle with a pre-configured curvature to a second target tissue to capture a second tissue sample.

20. A method for obtaining multiple tissue samples through an endoscope, the method comprising:
(i) advancing an endoscope comprising an outer sheath with multiple inner needles through a wall of a body lumen, each of said internal needles having a distinct pre-configured curvature and being retracted within the outer sheath;
(ii) upon reaching a target tissue site, extending one or more of said inner needles to multiple locations to capture multiple tissue samples; and
(iii) at least partially retracting one or more of said inner needles into said sheath.
